(19) United States
(12) Patent Application Publication
(10) Pub. No.: US 2020/0229966 A1
(43) Pub. Date: Jul. 23, 2020

(54) THERAPEUTIC DEVICE FOR INTRAORAL USE AND METHOD FOR OPERATING SUCH A THERAPEUTIC DEVICE

(71) Applicant: Bianca Berk, (US)

(72) Inventors: Bianca Berk, Marburg (DE); Jörg Köhler, München (DE); Gotthard Sauer-Sperling, Starnberg (DE)

(21) Appl. No.: 16/488,383

(22) PCT Filed: Feb. 26, 2018
(86) PCT No.: PCT/EP2018/054670

§ 371 Date: Aug. 23, 2019

(30) Foreign Application Priority Data
Feb. 24, 2017 (DE) 102017103950.3

(51) Int. Cl. A61F 5/56; A61B 5/00

(52) U.S. Cl. CPC A61F 5/566; A61B 5/4818; A61B 5/4836; A61B 5/038; A61B 5/4557; A61F 2005/563; A61B 5/682

(57) ABSTRACT

A therapeutic device for intraoral use for treatment of tooth grinding and/or tongue pressing and/or snoring, having:
at least two fastening devices for fastening the therapeutic device to at least one respective tooth of a user's upper jaw and/or lower jaw
at least one sensor device equipped to detect pressure load events and/or sound events and/or vibration events in the user's oral cavity;
at least one stimulation device for stimulating the user;
a control and/or regulating unit for controlled and/or regulated operation of the stimulation device as a function of a pressure load event and/or sound event and/or vibration event that occurs; and
a power supply
   wherein the fastening devices are connected to each other with at least one flexible connecting piece, which is equipped to rest against outsides of the teeth and/or insides of the teeth leaving uncovered the free tooth ends of the teeth.

### FIELD OF THE INVENTION

The invention relates to a therapeutic device for intraoral use and to an operating method for such a therapeutic device.

### BACKGROUND OF THE INVENTION

DE 10 2010 005 359 A1 has disclosed a night guard and a method for producing such night guards. Night guards of this have a guard profile with a U-shaped cross-section extending continuously along a dental arch, which can be placed onto the teeth of a dental arch. All of the free tooth ends along the curve of the guard are covered by the guard. The patent also discloses night guards, which are composed of singular fastening devices for placing onto molars. It also specifies, for example, an activation characteristic and a stimulation intensity.

DE 10 2004 009 883 has disclosed a device for treating the mandibular joint. Such a device has cushion-like, liquid-filled pads, which are connected to each other via an overflow line and with a different impingement of pressure on the cushions, and are intended to provide force compensation to the jaw. Such a device for treatment of the mandibular joint is not suitable for reducing, for example, tongue pressing or tooth grinding, if either of these is present to a pathological degree.

U.S. Pat. No. 4,976,618 has disclosed an intraoral sensor device, which has pressure tubes, which are positioned between teeth of the upper and lower jaw, and has a signal line for relaying such a pressure signal to an external device. The external device is placed outside the mouth. The external device is also connected to a speaker, which is embodied, for example, as an earbud. Such a sensor device is cumbersome to wear and often achieves only a low level of acceptance among corresponding users.

The object of the invention is to disclose a therapeutic device for intraoral use, which offers a high degree of wearing comfort and a high level of treatment success, particularly for reducing tongue pressing or tooth grinding or snoring and is found to be particularly comfortable and less annoying so that it can even be worn while sleeping.

Another object of the invention is to make it possible to make such a therapeutic device for intraoral use particularly inexpensive and simple to manufacture, particularly to disclose a therapeutic device, which fits a multitude of different users or can easily be adapted to the physiognomy of their oral cavities so that at least with regard to the geometry and three-dimensional shape of the therapeutic device, a single size and a single three-dimensional shape can be provided for a multitude of users.

### SUMMARY OF THE INVENTION

According to the invention, a therapeutic device for treatment for intraoral use, particularly of tooth grinding and/or tongue pressing and/or snoring, has at least the following:
at least two fastening devices for fixing the therapeutic device to at least one respective tooth of a user's upper jaw and/or lower jaw;
at least one sensor device embodied and equipped to detect pressure load events and/or sound events and/or vibration events in the user's oral cavity;
at least one stimulation device for stimulating the user if a pressure load event and/or a sound event and/or a vibration event occurs and
a control and/or regulating unit for controlled and/or regulated operation of the stimulation device as a function of a pressure load event and/or sound event and/or vibration event that occurs as well as
a power supply.

It is also characterized in that the fastening devices are connected to each other with at least one flexible connecting piece, which is equipped and embodied, with proper use of the therapeutic device, to rest against the outsides of the teeth and/or insides of the teeth while leaving uncovered the free tooth ends of the teeth along which the connecting piece extends, wherein in particular, with proper use of the therapeutic device, at least all of the above-mentioned components of the therapeutic device are positioned intraorally.

Such a therapeutic device for intraoral use ensures that with proper use, teeth of the upper jaw or lower jaw, depending on the jaw on which the therapeutic device is positioned, remain uncovered at their free ends. The user will find this to be comfortable compared to the prior art. The flexible connecting piece also particularly ensures that the holding devices, in their lateral spacing from one another, can, within broad limits, be adapted to the physiological boundary conditions of the user's oral cavity so that the therapeutic device can be easily adapted, for example, to different jaw geometries of different users.

Furthermore, particularly by means of the preferably prestressed contact of the connecting piece against either the outsides of the teeth (this, though, preferably when the therapeutic device is worn on the upper jaw) or the insides of the teeth (this, preferably when the therapeutic device is worn on the lower jaw) ensures a tighter seating of the therapeutic device in the user's mouth. Furthermore, an embodiment is possible in which regardless of the location where the therapeutic device is worn, an inner and/or outer connecting piece is provided.

The flexible connecting piece is reliably prevented from slipping into pain-sensitive areas, for example into areas of the gums.

Furthermore, the fact that the connecting piece rests against the free outsides of the teeth reliably prevents an intended slippage of the therapeutic device rearward in the user's mouth. When the connecting piece rests against the insides of the teeth of a jaw, this reliably prevents an unintended slippage of the therapeutic device toward the front. A double connecting piece can also be used, which extends on the outside of the teeth and the inside of the teeth.

Consequently, a secure positioning of the therapeutic device in the user's oral cavity is provided, even though in a particular embodiment of the invention, it is a “one size fits all” therapeutic device, i.e. at least one single size, which is embodied so that it fits the largest possible number of user jaws or user oral cavities. The therapeutic device according to the invention is consequently wearable and functional without its three-dimensional shape having to be individually adapted to the user's jaw during production, thus achieving an inexpensive mass production of the therapeutic device.

In another embodiment of the therapeutic device, the fastening devices are embodied as cap-like or U-shaped in cross-section like a U-rail and embrace at least one tooth, preferably a molar, when the therapeutic device is worn by the user. In this connection, it should be noted that according to the invention, the term “proper use” should be understood to mean that the therapeutic device and its components are equipped and embodied in such a way that when the user wears the therapeutic device properly and as intended, for example by sliding the therapeutic device onto teeth of the upper jaw or onto teeth of the lower jaw, the therapeutic device can perform its intended task and exhibit the intended properties. Thus proper use is to be understood as the use of the therapeutic device in a user's oral cavity in the slid-in state.

In another preferred embodiment of the therapeutic device, the therapeutic device has at least one wall section, which, with proper use, is positioned between teeth of the user's upper jaw and lower jaw and the at least one sensor device is integrated into this wall section.

According to the invention, the term “integrated” is understood to mean that the sensor device is either embedded in or constitutes the wall section that is positioned between the teeth of the upper jaw and lower jaw. It is likewise conceivable for the sensor device to be fastened so that it is positioned on this wall section or under this wall section.

Through such a placement, it is possible to reliably detect for example pressure events in the user's oral cavity of the kind that occur, for example, due to tooth grinding.

In another embodiment of the invention, the at least one sensor device is integrated into the connecting piece. In such an embodiment, particularly if the connecting piece extends along the insides of teeth, for example of the lower jaw, it is easily possible to detect a tongue pressing, which usually takes place due to an unwanted pressing of the tongue against the front teeth.

It is also alternatively or additionally possible for a signal line to be integrated into the connecting piece in order, for example, to connect a first pressure sensor device in one of the wall sections to a second pressure sensor device in another of the wall sections and/or to further connect it to the control and/or regulating unit.

In another embodiment, the control unit and/or the regulating unit and/or the stimulation device and/or the power supply is integrated or encapsulated, in particular cast into, the inside of a molded protrusion that is molded onto at least one of the fastening devices.

The stimulation devices can be embodied in the form of any type of suitable stimulators. In particular, vibrators such as unbalanced motors and/or piezoelectric oscillators are cited here. It is also possible to use stimulators in the form of electrodes, which produce an electric stimulation. It is furthermore possible to use stimulators in the form of a sound source, for example a speaker or the like.

In another embodiment of the therapeutic device, the molded protrusion is molded to correspond to a palatine arch or is embodied to be at least partially adaptable to a palatine arch or is adapted to a standardized palatine arch that corresponds to an average arch of the largest possible number of different users.

This measure increases the wearing comfort and the usability of the therapeutic device according to the invention for a large number of users with the same or a similar overall three-dimensional shape of the therapeutic device.

In another embodiment of the therapeutic device, the connecting piece is embodied as more flexible with regard to a curvature along the inside of the teeth or the outside of the teeth, i.e. around a vertical axis (H), than around a transverse axis (Q), which is oriented, for example, perpendicular to the shape of the connecting piece, in particular perpendicular to a broad side of the connecting piece. With a slightly looser seating of the therapeutic device in a user's oral cavity, this reliably prevents an unintended sagging of the connecting piece and thus an unwanted touching or chafing of the gums by the connecting piece.

In a particular embodiment of the therapeutic device, the connecting piece is composed of a rubber-like stretchable and/or compressible material and/or has an edge section that permits an accordion-like stretching and/or compressing. This embodiment makes it possible, in a particularly simple way, to insert the therapeutic device into the user's mouth in a slightly prestressed state from a mechanical standpoint. Particularly if there is no signal line extending through the connecting piece, this can constitute a particularly preferred embodiment. If a signal line extends through the connecting piece, then it must naturally be embodied so that it can follow along with corresponding stretching and compressing movements without being damaged.

In another embodiment of the therapeutic device, the connecting piece is embodied as tensile-elastic when the connecting piece is to be placed on the outside of the teeth in proper use and/or is embodied as pressure-elastic or compressible when the connecting piece is to be placed on the inside of the teeth in proper use.

These measures contribute to an improved wearing comfort and also to an improved sensing or detection of pressure events and/or sound events and/or oscillation events in the oral cavity since a defined and fixed seating of the therapeutic device in the oral cavity is provided.

Another object of the invention is to disclose an operating method for such a therapeutic device that uses an intraoral therapeutic device and is able to achieve a high degree of treatment success for reducing tooth grinding and/or tongue pressing and/or snoring. The operating method should also provide a plurality of adaptation options to user-specific or application-specific requirements.

Another object of the invention is to disclose an improved individual treatment by means of adjustable biofeedback.

This adjustability can be achieved, example, by means of software, e.g. by means of wireless transmission. This results in a flexible and non-invasive adjustability.

Another object of the invention is to disclose a therapeutic device for carrying out the method.

An operating method according to the invention for a therapeutic device for intraoral use provides the use of a therapeutic device for intraoral use. In particular, the treatment method according to the invention provides the use of a therapeutic device for intraoral use and has the following steps:
detecting at least one intraoral pressure event and/or oscillation event and/or sound event with a sensor device and generating a corresponding sensor output signal;
inputting the sensor output signal as an input signal into a control and/or regulating unit;
evaluating the input signal with the control and/or regulating unit and comparing the input signal to a predeterminable activation threshold;
activating a stimulation by means of a stimulator when the activation threshold is exceeded,
deactivating the stimulation when a predeterminable deactivation threshold is undershot;
wherein the use of a therapeutic device is carried out in which, before or during the operation of the device, at least one or more parameters selected from the group:
   input signal sensitivity of the control and/or regulating unit;
   output signal strength of the control and/or regulating unit;
   intensity of stimulation;
   type of stimulation;
   output signal strength of the sensor device;
   activation threshold;
   activation frequency;
   activation delay;
   deactivation threshold;
   hysteresis between the activation threshold and deactivation threshold
   is/are set in a predeterminable, definable way or is/are set and/or regulated in a variable way.

With the treatment method according to the invention, a higher level of treatment success can be expected. The treatment method according to the invention also enables a small, compact embodiment of the therapeutic device, which, with proper use of the therapeutic device, can in particular have an intraoral positioning of all of the components that are necessary for carrying out the operating method. Such an operating method is particularly suitable for reducing tooth grinding and/or tongue pressing and/or snoring and thus for reducing stress symptoms and/or headaches and/or tinnitus pathologies and/or other typical after-effects of the above-indicated undesirable actions.

Furthermore, the treatment method according to the invention enables a plurality of adaptation options to user-specific, i.e. personally-specific, requirements of the user, or to application-specific requirements, which particularly result from the disorders to be treated, for example nocturnal tooth grinding and/or tongue pressing and/or snoring.

In a particular embodiment of the method according to the invention, the setting of the parameters therefore takes place in a user-specific way, i.e. which is individual to the user, particularly with regard to physiological properties or boundary conditions such as sensitivity to stimulations, depth of sleep, body weight, and physique and/or in an application-specific way, i.e. as a function of the disorder that is to be treated, for example nocturnal tooth grinding, nocturnal tongue pressing, and/or snoring.

Another embodiment of the method according to the invention is characterized in that:
a) the input signal sensitivity of the control and/or regulating unit is increased when the patient has a more slender physique, for example, and is reduced when the user has a more muscular physique, for example, and/or
b) the output signal strength of the control and/or regulating unit and/or the intensity of the stimulation is increased when the user is a rather deep sleeper, for example, and is decreased when the user is a rather light sleeper, for example, and/or
c) the output signal strength of the sensor device is increased when the user is male, for example, and is decreased when the user is female, for example, and/or
d) the activation threshold is raised when the user has a more muscular physique, for example, or is a rather light sleeper and/or is lowered when the user has a more slender physique, for example, or is a rather deep sleeper and/or
e) the activation frequency is increased when the user is just at the start of the therapy, for example, and/or is decreased when at the end of the therapy, for example, the user is supposed to achieve as long-lasting a learning success as possible and/or
f) the activation delay is increased when at the end of the therapy, for example, the user is supposed to achieve as long-lasting a learning success as possible and/or is decreased when the user is just at the start of the therapy, for example, and/or
g) the deactivation threshold is raised when the user is just at the start of the therapy, for example, and/or is lowered when at the end of the therapy, for example, the user is supposed to achieve as long-lasting a learning success as possible and/or
h) the hysteresis between the activation threshold and the deactivation threshold is increased when the user is just at the start of the therapy, for example, or is a rather deep sleeper and/or is decreased when at the end of the therapy, for example, the user is supposed to achieve as long-lasting a learning success as possible or is a rather light sleeper.

In this context, “input signal sensitivity of the control and/or regulating unit” is understood to mean that with a lower input signal sensitivity of the control and/or regulating unit, with a certain input signal, a lower output signal is output and with a higher input signal sensitivity and the same certain input signal, a higher output signal strength is output by the control and/or regulating unit.

“Output signal strength of the control and/or regulating unit” is understood here to be a signal strength, which has a direct effect on the activation of the stimulator and thus a direct, but not necessarily proportional, influence on the intensity of the stimulation.

“Intensity of the stimulation” is understood to mean the strength of the stimulation, where different users can experience one and the same strength of stimulation with different intensities. Usually, the intensity of the stimulation is adjusted so that a sleep disturbance that is perceptible to the user does not occur or occurs to only a slight degree.

If the sensor device is an active sensor device such as a piezoelectric sensor, then its output signal can be influenced with regard to its output signal strength by providing suitable damping elements, for example, between the sensor device and the control and/or regulating unit.

The term “activation threshold” means a particular threshold of the input signal of the control and/or regulating unit or a signal that corresponds to this when it exceeds a particular value.

According to the invention, an “activation frequency” is understood to mean that a stimulation does not absolutely have to occur each time the activation threshold is exceeded. For example, it can be specified that an activation of the stimulation occurs only every other time, every third time, or every randomly selected number of times the activation threshold is exceeded.

For example, at the start of the treatment of a user, it is recommended for there to be a reaction by means of a stimulation in response to every pressure event, sound event, or oscillation event of the user, particularly also every grinding event of the user. In order to achieve the greatest possible and longest-lasting learning effect by means of a biofeedback embodied in this way, the frequency of the stimulation upon occurrence of corresponding events should taper off with use over time, for example after several successive sleep phases. For example, after a few days and/or weeks and/or months of use, there should no longer be a reaction to every grinding event. The events to which there should be a reaction can be selected by means of a random generator, for example. Furthermore, even over several successive uses, the stimulation can be changed, in particular reduced, in terms of its strength, i.e. also in terms of the intensity experienced by the user. Basically, each user reacts to stimulations differently and it is up to the user or to a person skilled in the art who is accompanying the treatment to individually select for the respective user the most successful treatment setting, i.e. parameter selection and/or activation sequence.

The term “activation delay” is understood to mean that a stimulation starts in a time-delayed fashion after the activation threshold is exceeded.

A “deactivation threshold” is understood to mean an additional limit value for the input signal of the control and/or regulating unit, the undershooting of which causes a currently occurring stimulation to be embodied.

Between the deactivation threshold and the somewhat higher activation threshold a hysteresis logically occurs, whose magnitude between the activation threshold and the deactivation threshold is adjustable. This hysteresis can, for example, be selected to be greater if the user does indeed react to a stimulation, but this reaction results only slowly in a reduction of the disorder to be treated. In such a case, if the deactivation threshold is slightly lower, then a slow decrease of the disorder thus results in a longer stimulation.

With this plurality of adjustable parameters, the operating method according to the invention can be adjusted within broad limits to personal/individual requirements of the user or to disorder-specific requirements of disorders that are to be treated.

In another embodiment of the treatment method according to the invention, if the parameter or parameters are set before and/or during the treatment as values that are chronologically constant for the duration of the treatment or the parameter or parameters are variably set before and/or during the treatment as values that are chronologically variable depending on another variable, e.g. as a function over time and/or over another variable such as ambient noise level or a variable of the like.

In another embodiment of the method according to the invention, the parameter or parameters are set before or during the operation as a function of body parameters and/or body features of the user, for example as a function of the respiratory rate, the body temperature, the pulse, the physical activity of the user, for example during sleep, and/or the intraorally sensed pressure, the intraorally sensed oscillation, or the intraorally sensed sound. In this connection, the parameter or parameters are set and/or regulated for example statically or variably, i.e. continuously adapted to the corresponding variable.

### BRIEF DESCRIPTION OF THE DRAWINGS

In the following, the invention will be explained in greater detail based on the drawings. In the drawings:

FIG. 1: shows a very schematic perspective view of a first embodiment of the therapeutic device for intraoral use in the state in which it has been slid onto a jaw of the user and thus in the state of proper use;

FIG. 2: shows a top view of the therapeutic device according to FIG. 1;

FIG. 3: shows a top view of a second embodiment of the therapeutic device according to the invention for intraoral use;

FIG. 4: shows a very schematic perspective view of a third embodiment of the therapeutic device according to the invention for intraoral use;

FIG. 5: shows a perspective, very schematic view of a fourth embodiment of the therapeutic device according to the invention for intraoral use.

### DETAILED DESCRIPTION OF THE PREFERRED EMBODIMENTS

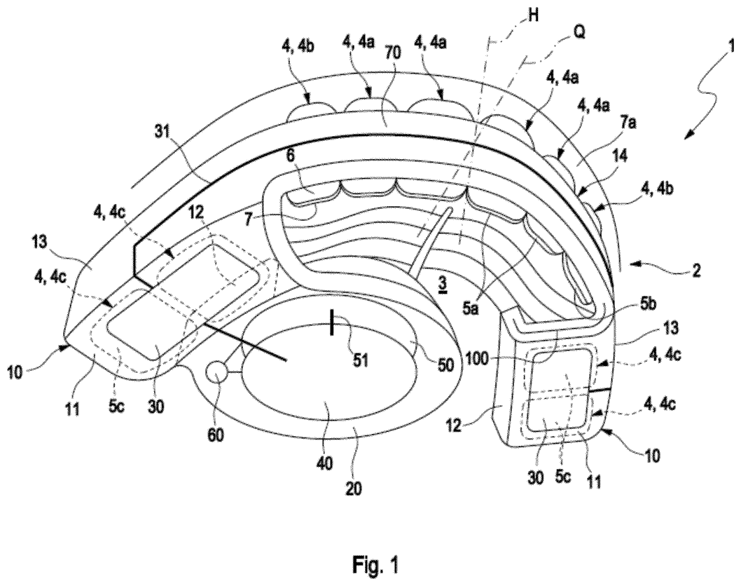

A therapeutic device 1 according to the invention for intraoral use is schematically depicted in FIGS. 1 in a first embodiment.

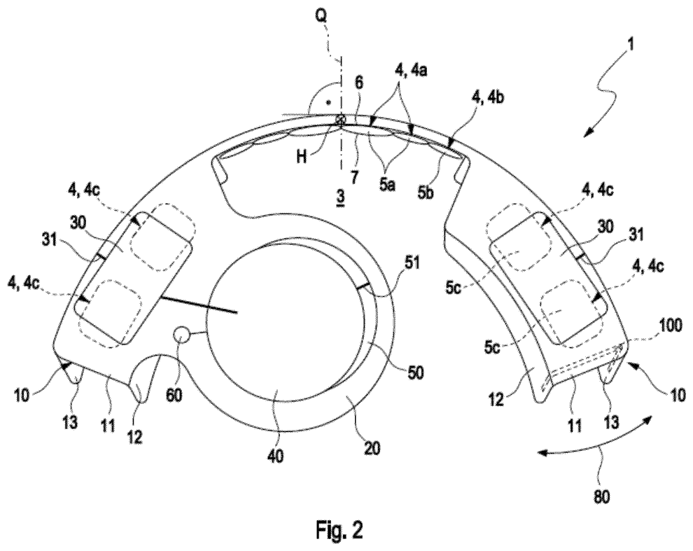

FIG. 1 shows a perspective depiction of the therapeutic device 1 that has been inserted and slid onto a user's jaw 2. The exemplary embodiment according to FIGS. 1 and 2 shows the therapeutic device 1 slid onto the upper jaw of the user, which is indicated by the schematic depiction of a palate 3. The jaw 2, in this case the upper jaw, has teeth 4. In particular, incisors 4a, canines 4b, and molars 4c are schematically depicted. The teeth 4 have free ends 5; free ends of the incisors 5a, free ends of the canines 5b, and free ends of molars 5c are indicated. The teeth 4 have outsides 6 and insides 7 that are oriented toward the palate 3 or a palatine arch of the palate 3.

The gums 7a are also schematically depicted.

The therapeutic device 1 for intraoral use has two instating devices 10, which are embodied as U-shaped in cross-section in a form similar to U-rails and are also equipped and embodied to be slid onto at least one molar 4c, preferably several molars 4c, embracing them. The fastening devices in this case have at least one wall section 11, which, with proper use of the therapeutic device 1, covers the free ends 5c of the molars 4c so that this wall section 11, with proper use of the therapeutic device, is subjected to a crushing between the molars of the upper jaw and corresponding molars of the lower jaw when the user bites down.

In addition, the fastening devices 10 have inner wall sections 12, which rest against the molars 4c on the inside, i.e. on the palatal side, preferably in a clamping fashion.

The fastening devices also have outer wall sections 13, which rest against the outsides of the molars 4c so that the wall sections 11, 12, 13 embrace at least one molar in clamp fashion and thus function as a fastening device 10 for the therapeutic device 1.

The fastening devices 10 are preferably positioned on opposing molars 4c of a dental hemiarch. The fastening devices are connected to each other with at least one flexible connecting piece 14; in proper use, the connecting piece 14 extends along the outsides 6 (shown in FIG. 1) of the incisors 5a and possibly the canines 5b, and rests against this region, particularly in a prestressed way. In this case, the connecting piece 14 does not cover the free ends of the incisors 5a, canines 5b, and any molars 5c not covered by the wall section 11. The connecting piece 14 is also positioned and embodied in such a way that with proper use, there is no contact, if at all possible, with the gums 7a.

In addition, the therapeutic device 1 has a molded protrusion 20, which is for example integrally joined to or formed onto one of the fastening devices 10. It is preferable for the molded protrusion 20 to be formed onto only one of the fastening devices 10 so that by means of the flexible connecting piece 14, the other fastening device 10 can be moved relatively easily relative to the fastening device 10 with the molded protrusion 20 so that a good adaptability to different jaw geometries of different users can be achieved. In other words, by means of the flexible, in particular resiliently flexible, embodiment of the connecting piece, the lateral distance between the fastening devices 10 can be adapted within broad limits to the physiological circumstances in a user's oral cavity.

A sensor device 30 is embedded or positioned in the wall section 11 that is positioned between the molars 5c of the upper jaw and molars of a lower jaw when a user bites down and is thus subjected to a crushing. The sensor device 30 can, for example, be a pressure sensor, e.g. a force-sensing resistor. It can, however, also easily be embodied as an active pressure sensor, e.g. as a piezoelectric sensor, which outputs an electrical voltage when it is deformed by the exertion of a force. In the simplest case, the sensor device 30 can also be embodied as a pressure switch, which can reflect only a switched-on state and a switched-off state. Preferably, however, the sensor devices 30 are embodied in such a way that a sensor signal is produced, which changes as a function of the pressure applied. Such a change can occur proportionally or in accordance with a different function, e.g. logarithmically.

Basically, it is sufficient to provide such pressure sensor device 30 in just one wall section 11 of one of the two fastening devices 10. It is preferable, however, to provide such sensor devices 30 in both fastening devices 10 in order to provide a reliable sensing of pressure events or the like even when there is an uneven load on the dental arches, for example due to an oblique bite.

For example, if such a sensor device 30 is provided in each fastening device 10, then it is advisable to provide corresponding sensor signal lines 31 for connecting to the sensor device 30 that is not positioned in the vicinity of the molded protrusion 20. Such a signal line 31 can be suitably provided along the connecting piece 14, in particular embedded in it.

The sensor devices 30 are connected to a control/regulating unit 40, which is equipped and embodied to receive and evaluate sensor signals of the at least one sensor device 30.

The therapeutic device for intraoral use also has a stimulation device 50, which can be activated by the control/regulating unit 40 via a schematically indicated connection 51. The stimulation device 50 can, for example, be a mechanical and/or electrical stimulation device. The stimulation device can also be embodied as a thermally acting stimulation device or a stimulation device that functions as a sound source. It is conceivable to use any mechanically embodied stimulation device, for example unbalanced motors and/or piezoelectric oscillators. Electrically acting stimulation devices can, for example, be stimulating electrodes, which, with proper use of the therapeutic device 1, contact the user's gums or palate or buccal tissue in order to activate electrical stimulus there as needed.

A thermally acting stimulation device 50 can, for example, be a heating element or cooling element, e.g. a Peltier element, which produces a hot and/or cold stimulus in particular locations, for example on a user's palate or gums.

Naturally, the stimulation device 50 can also be embodied as a sound source in order to produce an acoustic stimulation of the user, for example by producing a beeping or humming noise.

The therapeutic device 1 also has a power supply 60, which is used to supply power to the control/regulating unit, the stimulation device(s), and the sensor devices as needed.

For example, the above-described embodiment of the therapeutic device 1 is suitable for detecting an involuntary tooth grinding while a user sleeps and to treat it with suitable stimulation, particularly to the effect that a reduction, possibly even a prevention of tooth grinding takes place based on a biofeedback and based on a learning process for the user that results from this, which can even take place subconsciously.

If the therapeutic device 1 is used to combat involuntary snoring or the like, then the sensor devices can also be embodied in the form of microphones for recording sound events or other oscillation events in the oral cavity.

It can likewise be useful to provide corresponding sensor devices 30 such as pressure sensors or the like at locations in which a so-called tongue pressing takes place, i.e. a convulsive pressing of the tongue during sleep against a particular location in the oral cavity, e.g. the insides of the teeth 7. Such a tongue pressing can be so extensive that over time, it even changes the position of the teeth 4 in the jaw. In many cases, tongue pressing often leads to overexertion reactions of the corresponding musculature and possibly to headaches, neck pain, or other user complaints.

It is also stipulated that with proper use, at least the fastening devices 10, the connecting piece 14, the molded protrusion 20 containing the control/regulating unit and the stimulation device as well as the sensor devices 30 and possibly provided signal lines 31 and the power supply 60 are all positioned intraorally, which makes it unnecessary, for example, for wires and/or antennae and/or other such components to be routed out from the mouth. Users often find this to be very uncomfortable and also unattractive. A complete intraoral placement of the therapeutic device 1 counteracts these properties, which are found to be disadvantages, of therapeutic devices from the prior art.

Basically, the connecting piece 14 can have a cross-sectional three-dimensional shape of any kind. It is particularly preferable, however, for a cross-sectional shape of the connecting piece 14 to be selected, which is embodied as more flexible with regard to a bending around a vertical axis H than around a transverse axis Q. The vertical axis H in this case is understood to mean that it is oriented approximately parallel to the longitudinal dimensions of the teeth from the gums to their free ends 5a. A transverse axis Q is understood to mean that it is oriented for example perpendicular to an outside of the teeth 16 and thus perpendicular to an outside 70 of the connecting leg or a corresponding inside. The essential thing is that the different flexibility in a direction around the vertical axis H (more flexible embodiment) by contrast with a bending around an axis Q results in the fact that the connecting piece 7 is embodied as stable in a vertical direction and reliably continues to rest against the outsides of the teeth 6. On the one hand, this ensures a good seating of the therapeutic device 1 in the oral cavity. On the other hand, this also reliably prevents the connecting piece 14 from chafing against the gums 7a, for example, and thus possibly causing an irritation of the gums 7a. Users can be expected to find this to be comfortable.

FIG. 2 schematically depicts a top view of the therapeutic device 1 according to FIG. 1. The vertical axis H in this case is depicted as an axis that extends perpendicularly out from the plane of the drawing or down into it. The transverse axis Q is schematically depicted as extending at right angles to the outside or broad side 70 of the connecting piece 14.

It is also clear that the free tooth ends 5 of at least the incisors and possibly the canines remain free.

A therapeutic device 1 like the one described above can naturally be inserted and used on both an upper jaw and a lower jaw. It is particularly advantageous that one of the fastening devices 10 (the fastening device 10 shown on the right in FIG. 2) is connected to the other fastening device 10 and the molded protrusion 20 only by means of the connecting piece 14, which enables an easy mobility of the first fastening device 10 relative to the other fastening device 10 in a schematically depicted double-arrow direction 80. By means of this, it is easily possible to adapt the therapeutic device to different jaw geometries of the user.

This particularly opens up the possibility of mass producing a therapeutic device according to the invention 1 as a standardized therapeutic device since an individual adaptation of such a therapeutic device to specific circumstances, in particular geometrical circumstances, in the oral cavity of a patient is possible due to the structural embodiment of the therapeutic device. Consequently, such therapeutic devices can be manufactured in only one basic size or at least in only a few different basic sizes, in order to be usable for a multitude of different users.

It can also be useful, if necessary, to embody the wall sections 12 and 13 as flexible or deformable relative to each other so as to ensure an adaptation to different molar widths and thus a fixed seating. For example, this can be achieved if spring clips 100 or for example deformable metal strips are provided in the fastening devices 10.

Furthermore, medically harmless plastics have proven valuable as materials for the therapeutic device, at least for the fastening devices 10, the connecting piece 14, and the molded protrusion 20. Such a therapeutic device 1 can, for example be manufactured by means of deep drawing (more suitable for small-scale production) or by means of injection molding.

Particularly for mass production, it is suitable for the therapeutic device 1 to have a design that is conducive to injection molding. In particular, the sensor devices, signal lines, control/regulating unit, stimulation unit, power supply, and the like are cast into the therapeutic device 1 during the injection molding or are embedded into it in some other way. Possible materials for producing the therapeutic device 1 and for casting-in the other components can, for example, include medically harmless silicones, plastics, and/or soft plastics.

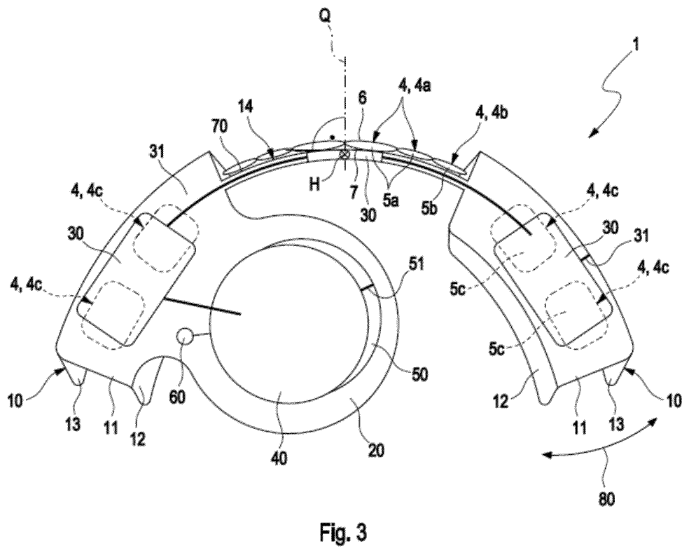

FIG. 3 shows a second embodiment of the therapeutic device according to the invention 1. It differs from the above-described embodiment essentially due to the fact that the connecting piece 14, with proper use of the therapeutic device 1, is positioned on the insides 7 e.g. of the incisors and/or canines 4a, 4b, leaving their free ends 5a, 5b uncovered, in particular rests against them in a prestressed way, e.g. in a compressed way. In an embodiment of this kind that is particularly suitable for use of the therapeutic device 1 on the lower jaw, a sensor device 30 can be advantageously integrated into the connecting piece 14 in order, for example, to detect a tongue pressing when a tongue presses against the teeth from the inside.

In the case of a use of the therapeutic device 1 on the lower jaw, allowing the connecting piece 14 to extend along the insides of the teeth is also advantageous since when biting down, a physiologically normal overbite of the upper teeth outside the lower teeth takes place. The connecting piece 14 is thus in a region that does not obstruct the biting. The same is conversely true for the use of the therapeutic device 1 on the upper jaw. There, it is particularly advantageous to allow the connecting piece 14, with proper use, to extend along the fee outsides 6 of the upper jaw in order to prevent the connecting piece 14 from getting between the teeth of the upper jaw and the corresponding incisors or canines of the lower jaw during biting.

With the therapeutic device according to the invention, a therapeutic device is produced in a simple way, which is based on the effect of so-called biofeedback and therefore permits the expectation of good learning effects and treatment successes. The therapeutic device according to the invention can easily be adapted to a wide variety of indications (tooth grinding, tongue pressing, and/or snoring, etc.).

In a particularly preferred embodiment, the therapeutic device 1 is embodied as an adjustable and configurable, in particular regulatable, biofeedback system, which particularly permits the stimulation and the activation sensitivity and other parameters, as described further below in a treatment method according to the invention, to be adapted in a way that is individual to the user or to be individually adapted to different indications in an application-specific way.

Although the above-described therapeutic device is particularly suited for mass production and fits most users or can be easily adapted to them, the idea on which the invention is based can naturally also be transferred to and easily used in therapeutic devices that are produced individually for the user or the application field.

In another embodiment of the invention, additional memory elements can be provided in order, for example, to record the frequency and intensity of tooth grinding, tongue pressing, or snoring and the corresponding reactions of the user to the applied stimulation. Naturally, corresponding reading devices can also be provided for this, for example Bluetooth interfaces, infrared interfaces, or the like.

There is also the possibility for the stimulation to be regulated and/or controlled as a function of particular body parameters or body properties or body features of the user. In such a case, the scope of the invention naturally includes providing corresponding sensors such as sensors for the body temperature, respiratory rate, blood pressure, pulse, and the like or to provide these in the form of external sensors that communicate with the control and regulating unit, either by wire or by radio, so that such control variables can be correspondingly processed by the control/regulating unit.

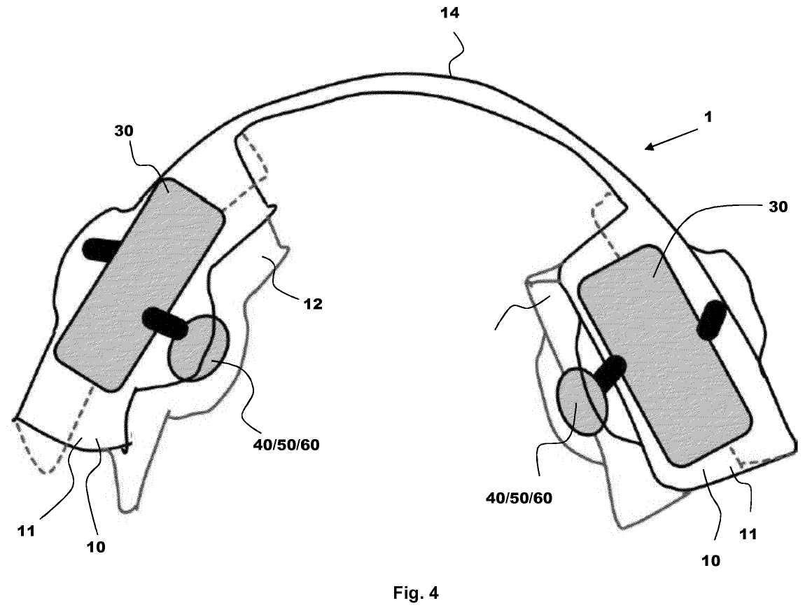

A third embodiment of the therapeutic device according to the invention 1 is shown in FIG. 4. This embodiment of the therapeutic device has a plurality of components/functions that are the same and that are also present in the embodiments described above. To this extent, parts/components that remain the same have been provided with the same reference numerals. The preceding description applies correspondingly here for the present embodiment.

In order to leave the palate of the user of the therapeutic device 1 uncovered, the present embodiment proposes embedding the control and regulating unit 40, a stimulation device 50, and the power supply 60 in the inner wall section 12 of the fastening devices 10. In this case, a respective control/regulating unit 40, a stimulation device 50, and a power supply 60 can be positioned, for example, in each inner wall section 12 of each fastening device 10. In such a case, each side, i.e. the left side and right side, of the therapeutic device 1 can operate independently. A connection between the left side and the right side can be omitted so that no signal line 31 has to be embedded in the connecting piece 14.

In the embodiment shown according to FIG. 4, a system composed of the control/regulating unit 40, the stimulation device 50, and the power supply 60 can be embedded in the inner wall section 12, i.e. toward the user's tongue or toward the user's palate.

In spite of this, the components 40, 50, 60 can naturally also be embedded in the outer wall section 13, i.e. on the buccal side in the outer section of the user's jaw.

This measure particularly provides the user with a large amount of open space for the tongue and in the region of the palate, which is often found to be comfortable.

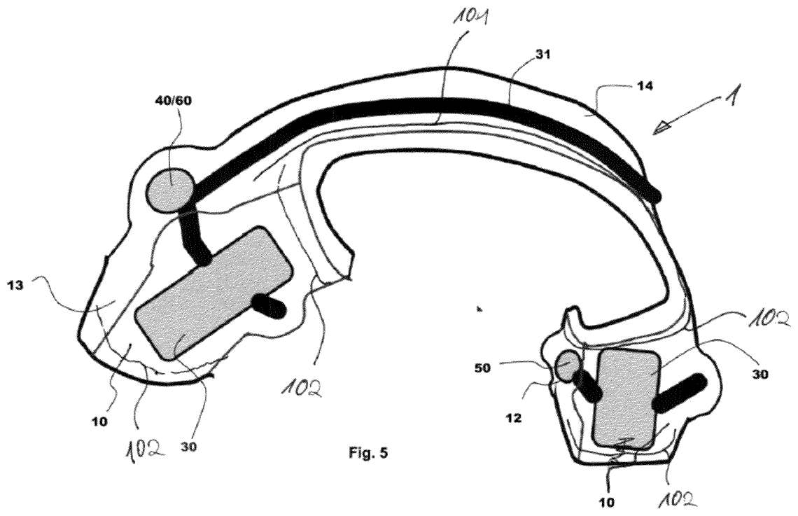

Another embodiment of the therapeutic device according to the invention is schematically depicted in FIG. 5. The therapeutic device 1 in the embodiment according to FIG. 5 shows a variant in which a signal line 31 is embedded in the connecting piece 14. A stimulation device 50 is embedded, for example, in the inner wall section 12 of the first fastening device 10. A power supply 60 and a control/regulating unit 40 are, for example, positioned in the outer wall section 30 of the second fastening device 10. Naturally, in addition to the explicitly shown and described positioning variants, all other variants are also conceivable. Thus, for example, the stimulation device 50 can absolutely also be positioned in the outer wall section of a fastening device 10, while the control/regulating unit 40 and the power supply 60 can be positioned or embedded in an inner wall section 12 of the other fastening device 10. The present variants can be adapted to the needs of different patients. A placement of the components 40/50/60 in outer wall sections 13 is particularly suitable, for example, in patients with very narrow dental arches, who have less room in the interior space between the rows of teeth.

An embodiment of the therapeutic device 1 according to FIG. 5 is also particularly suitable for patients/users who find it uncomfortable to have an object resting against the palate.

FIG. 5 also graphically depicts yet another optional additional feature of the therapeutic device, which naturally can also be used on all of the embodiments described above, e.g. those shown in FIGS. 1, 2, 3, and 4. This additional feature is not graphically depicted therein, however.

This additional feature relates alternatively or cumulatively to a first reinforcing device 101 and to other second reinforcing devices 102. For example, these devices, which are referred to as reinforcing devices 101 and 102, can be plastically deformable wires or bending bars with cross-sections that are embodied as other than round, which are embedded into the carrier material of the corresponding regions of the therapeutic device 1 and therefore as much as possible, do not come directly into contact with the user's body. The first reinforcing device 101, for example, is a wire or other reinforcing device, e.g. a flat wire, which extends along the curve of the connecting piece 14 adjacent to the signal line 31 (if provided). This reinforcing device 101 can easily be bent into shape by the patient so that the embodiment of the connecting piece 14, which is intrinsically relatively pliable, gains a certain degree of rigidity and as a result, the entire therapeutic device 1 can be adapted to the individual circumstances (radius of the user's dental arch) and be set in a correspondingly flexible way.

The second reinforcing devices 102 basically function in the same way. They can likewise be made of plastically deformable devices such as round wires or flat wires by means of which the width/breadth of the fastening devices 10 can be adjusted through plastic bending of the fastening devices 10 so that it is possible to achieve a better clamping of the therapeutic device 1 to the user's molars. Several reinforcing devices 102 can also be provided in each fastening device 10, for example viewed from the sensor devices, one closer to the incisors and one closer to the back molars so that over their entire length, the shape of the fastening devices 10 can be better adapted to the user's individual circumstances.

These additional solutions (reinforcing devices 101 and 102) therefore permit an improved adaptation of the therapeutic device 1 to the individual needs of the user. The rigidity of the therapeutic device 1 as a whole is also improved so that the fit is further improved and it holds onto the teeth better. A suitable flat wire for use in the connecting piece 14 has a length, for example of between 60 mm and 85 mm, a height of 1.5 mm to 3 mm, and a thickness of 0.2 mm to 0.5 mm.

If need be, it can also be advantageous, as a reinforcing device 101, to provide a plastically bendable support for the connecting line 31 so that the reinforcing device 101 and the connecting line 31, before being embedded into the support material of the therapeutic device 1 (e.g. silicone), are embodied as a single component that can be plastically deformed by bending.

It is also conceivable for the reinforcing devices 102, which are provided for embedding in the fastening devices 10, to already be mechanically connected to other components such as the sensor device before being embedded into the support material of the therapeutic device 1, thus facilitating the embedding process. These measures constitute a simplification of the manufacture of the therapeutic device 1.

To further improve the fit, alternatively to the above measures or cumulatively with them, it is also possible for the user at a dentist or the user himself to introduce a so-called lining silicone under the U-shaped fastening devices 10 and adapt this “lining silicone” to the user's tooth shape, in particular the user's crown shape (e.g. by “biting into it”). It is thus possible to produce a plateau on which the fastening device 10 rests over a large area and not just at certain points so that the individual fit is improved further. Where appropriate, it can make sense for “lining silicone” that has been adapted to the individual patient to be subsequently attached firmly to an underside of the fastening device 10 inside the U-shaped structure by means of gluing or in another suitable way.

1. A therapeutic device for intraoral use for treatment of tooth grinding and/or tongue pressing and/or snoring, comprising:
at least one sensor device embodied and equipped to detect pressure load events and/or sound events and/or vibration events in a user's oral cavity;
at least one stimulation device for stimulating the user if a pressure load event and/or a sound event and or a vibration event occurs;
a control and/or regulating unit for controlled and or regulated operation of the stimulation device as a function of a pressure load event and/or sound event and/or vibration event that occurs as well as;
a power supply; and
at least two fastening devices for fastening the therapeutic device to at least one respective tooth of the user's upper jaw and or lower jaw, wherein the fastening devices are connected to each other with at least one flexible connecting piece, which is equipped and embodied, with proper use of the therapeutic device, to rest against outsides of the user's teeth and/or insides of the user's teeth leaving uncovered the free tooth ends of the teeth, with at least all of the above-mentioned components of the therapeutic device being positioned intraorally with proper use of the therapeutic device: and the at least one sensor device and or a signal line is integrated into the at least one connecting piece, or each of the at least one sensor devices is associated with a control/regulating unit, a stimulation device, and a power supply so that each fastening device has an independently functioning system composed of a sensor device, a control/regulating unit, a stimulation device, and a power supply.

2. The therapeutic device according to claim 1, wherein the at least two fastening devices are embodied as cap-like or U-shaped in cross-section like a U-rail and are embodied to embrace at least one tooth.

3. The therapeutic device according to claim 1, wherein the therapeutic device has at least one wall section which, with proper use, is positioned between teeth of the user's upper jaw and lower jaw and the at least one sensor device is integrated into this wall section.

4. (canceled)

5. The therapeutic device according to claim 1, wherein the control unit and or the regulating unit and/or the stimulation device is integrated or encapsulated, in particular cast into, an inside of a molded protrusion that is molded onto at least one of the fastening devices.

6. The therapeutic device according to claim 5, wherein the molded protrusion is molded to correspond to a palatine arch and/or is embodied to be at least partially adaptable to a palatine arch and/or is adapted to a standardized palatine arch that fits a largest possible number of different users.

7. The therapeutic device according to claim 1, wherein the at least one connecting piece is more flexible with regard to a curvature along an inside of the teeth or an outside of the teeth, i.e. around a vertical axis, than around a transverse axis oriented perpendicular to a broad side of the at least one connecting piece.

8. The therapeutic device according to claim 1, wherein the at least one connecting piece is composed of a rubber-like stretchable and/or compressible material and/or has an edge section that permits an accordion-like stretching and/or compressing.

9. The therapeutic device according to claim 1, wherein the at least one connecting piece is tensile-elastic when the at least one connecting piece is placed on the outside of the teeth with proper use and/or is pressure-elastic or compressible when the at least one connecting piece is placed on the inside of the teeth with proper use.

10. The therapeutic device according to claim 1, wherein the therapeutic device does not have a palatal-side molded protrusion and the control/regulating unit and/or the stimulation device and/or the power supply are embedded or positioned in one or more inner wall sections or one or more outer wall sections.

11. (canceled)

12. The therapeutic device according to claim 1, wherein the at least one connecting piece does not have a signal line.

13. The therapeutic device according to claim 1, wherein in a region of the at least one connecting piece and/or in a region of the at least two fastening devices, one or more plastically flexible, deformable reinforcing devices are provided.

14. The therapeutic device according to claim 13, wherein the reinforcing devices are a plastically deformable wire or flat wire.

15. An operating method for using the therapeutic device according to claim 1, comprising the following steps:
detecting at least one intraoral pressure event and/or oscillation event and/or sound event with the at least one sensor device and generating a corresponding sensor output signal;
inputting the sensor output signal as an input signal into the control and/or regulating unit;
evaluating the input signal with control and/or regulating unit and comparing the input signal to a predeterminable activation threshold;
activating a stimulation using the at least one stimulation device when the activation threshold is exceeded;
deactivating the stimulation when a predeterminable deactivation threshold is undershot;
wherein use of the intraoral therapeutic device is carried out in which, before or during operation of the device, at least one or more parameters selected from the group consisting of:
input signal sensitivity of the control and/or regulating unit;
output signal strength of the control and/or regulating unit;
intensity of the stimulation;
type of stimulation;
output signal strength of the at least one sensor device;
activation threshold;
activation frequency;
activation delay;
deactivation threshold;
hysteresis between the activation threshold and the deactivation threshold
is/are set in a predeterminable, definable way or is/are set and/or regulated in a variable way.

16. (canceled)

17. The method according to claim 15, wherein:
a) the input signal sensitivity of the control and/or regulating unit is increased when the patient has a more slender physique; and is reduced when the user has a more muscular physique, and/or
b) the output signal strength of the control and/or regulating unit and/or the intensity of the stimulation is increased when the user is a rather deep sleeper, and is decreased when the user is a rather light sleeper; and/or
c) the output signal strength of the sensor device is increased when the user is male, and is decreased when the user is female; and/or
d) the activation threshold is raised when the user has a more muscular physique, or is a rather light sleeper, and/or is lowered when the user has a more slender physique, or is a rather deep sleeper; and/or
e) the activation frequency is increased when the user is at a start of a therapy, and/or is decreased when, at an end of the therapy, the user is supposed to achieve as long-lasting a learning success as possible; and/or
f) the activation delay is increased when, at the end of the therapy, the user is supposed to achieve as long-lasting a learning success as possible, and/or is decreased when the user is at the start of the therapy; and/or
g) the deactivation threshold is raised when the user is at the start of the therapy, and/or is lowered when, at the end of the therapy, the user is supposed to achieve as long-lasting a learning success as possible; and/or
h) the hysteresis between the activation threshold and the deactivation threshold is increased when the user is at the start of the therapy or is a rather deep sleeper, and/or is decreased when, at the end of the therapy, the user is supposed to achieve as long-lasting a learning success as possible or is a rather light sleeper.

18. (canceled)

19. The method according to claim 15, wherein the parameter or parameters are set or regulated before or during the operation as a function of body parameters and/or body features of the user.

20. A therapeutic device suitable for carrying out the method according to claim 15.
